(19) United States
(12) Patent Application Publication
(10) Pub. No.: US 2006/0088482 A1
(43) Pub. Date: Apr. 27, 2006

(54) Oral and dental care agent

(76) Inventors: Peter Wulknitz, Leichlingen (DE); Han Laska, Duesseldorf (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/224,606

(22) Filed: Sep. 12, 2005

Related U.S. Application Data
(63) Continuation of application No. PCT/EP04/02503, filed on Mar. 11, 2003.

(30) Foreign Application Priority Data
Mar. 12, 2003 (DE) 103 11 171.9

(51) Int. Cl. A61K 8/55

(52) U.S. Cl. 424/49

(57) ABSTRACT

The invention relates to oral and dental care agents, containing: an active ingredient combination (A) of at least one cationic component with an anti-bacterial action, (B) at least one active ingredient with an anti-tartar action, selected from the group of azacycloalkane diphosphonic acids or their phnysiologically compatible salts and (C) at least one binding agent, selected from xanthan gum, carboxymethylcellulose or a mixture of said two components. The oral and dental care anti-bacterial agents do not cause the discoloration associated with chlorohexidine and reduce or prevent discoloration to human teeth. This is unique for oral and dental care agents based on chlorohexidine. The inventive oral and dental care agents also have a tartar prevention and plaque reducing agent.

### CROSS-REFERENCE TO RELATED APPLICATIONS

This application is a continuation under 35 U.S.C. § 365(c) and 35 U.S.C. § 120 of International Application PCT/EP2004/002503, filed Mar. 11, 2004. This application also claims priority under 35 U.S.C. § 119 of DE 103 11 171.9, filed Mar. 12, 2003, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

This invention relates to oral and dental care preparations based on a special combination of active components which can contribute towards reducing and inhibiting discoloration on human teeth and towards inhibiting scale and plaque.

Modern oral and dental care preparations are used above all for cleaning and freeing the tooth surface from food remains, discoloration and firmly adhering plaque. Plaque in particular is the basis for the formation of scale which in turn results in the development and spread of gum inflammation, bleeding (gingivitis) and caries.

Accordingly, to keep the teeth and gums healthy, the bacterial coating has to be quantitatively removed continuously by corresponding dental care. Ideally, an oral and dental care preparation should even completely inhibit the formation of plaque and scale through special additives.

In the past, various efforts have been made to solve the problems mentioned above. For example, it is known from the literature that certain antibacterial components, as additives in toothpastes and mouthwashes, are capable of counteracting inflammation of the gums. In this connection, chlorhexidine has long been known as a very effective antibacterial agent. The effect of chlorhexidine is based on its high retention level in the oral cavity which is equivalent to a certain slow-release effect or long-term effect. Hitherto, however, the problem with chlorhexidine used over a long period has been the formation of discoloration in the mouth which is caused by chlorhexidine-induced deposits of, above all, the polyphenols present in various foods, such as tea, coffee and red wine. This discoloration has an unattractive effect in particular on the teeth and are generally not accepted by consumers. Accordingly, despite its considerable effectiveness in oral and dental care preparations, chlorhexidine is not normally used in the long term.

(2) Description of Related Art, Including Information Disclosed Under 37 C.F.R. §§ 1.97 and 1.98.

In EP 304 627 B1, an attempt was made to tackle the problem of discoloration by a synergistic combination of chlorhexidine and an alkyl polyglycoside. Through the synergistic increase in effect of the two components, the chlorhexidine content could be reduced to such an extent that the problem of discoloration was clearly reduced. However, no breakthrough was made in the more effective elimination or inhibition of plaque.

An attempt to reduce scale and plaque was made in EP 480 811 A1 by combining a cationic antibacterial agent with an anti-scale agent (preferably an azacycloalkane disphosphonic acid salt). However, the problem of discoloration could not be solved by regular use of the preparation on account of the high concentrations of chlorhexidine.

Accordingly, there is still a need for oral and dental care preparations which would be capable of inhibiting and/or reducing the formation of plaque and scale without having to tolerate the disadvantages of the prior art, such as discoloration, for example.

### BRIEF SUMMARY OF THE INVENTION

Now, it has been found, completely surprisingly, that not only a discoloration-reducing and—inhibiting effect, but also a strong scale-inhibiting effect and an antiplaque effect can be obtained by a special combination of active components.

Accordingly, the present invention relates to oral and dental care preparations containing an active-component combination of
(A) at least one cationic antibacterial component,
(B) at least one antiscale component selected from the group of azacycloalkane diphosphonic acids or physiologically compatible salts thereof and
   at least one binder selected from xanthan gum, carboxymethyl cellulose or a mixture of these two components.

### BRIEF DESCRIPTION OF THE SEVERAL VIEWS OF THE DRAWING(S)

Not Applicable

### DETAILED DESCRIPTION OF THE INVENTION

In the context of the invention, cationic antibacterial components are understood to be bisbiguanide derivatives. The compounds chlorhexidine and alexidine are particularly preferred. These two compounds are preferably used in the oral and dental care preparations in the form of their water-soluble, physiologically compatible salts. Such salts are, for example, the acetates, gluconates, hydrochlorides, hydrobromides, citrates, bisulfites, fluorides, sorbates, salicylates, maleates, tartrates, fumarates, ethylenediaminotetra-acetates, iminodiacetates, cinnamates, thiocyanates, arginates, benzoates and glutarates of chlorhexidine or alexidine.

Other antibacterial biguanide compounds suitable for use in accordance with the invention are, for example, polyhexamethylene biguanide compounds of the Vantocil® IB type (ICI), the 1,6-bis-(4-chlorobenzylbiguanido)-hexane (fluorhexidine) known from DE-A-1 964 196 and the antibacterial biguanide compounds known from U.S. Pat. No. 2,684,924, U.S. Pat. No. 2,990,425, U.S. Pat. No. 3,468,898, U.S. Pat. No. 4,022,834, U.S. Pat. No. 4,053,636 and U.S. Pat. No. 4,198,392.

In a most particularly preferred embodiment of the invention, chlorhexidine digluconate is used as the antibacterial component in the oral and dental care preparations.

The antibacterial component (A) is used in the oral and dental care preparations according to the invention in quantities of 0.001 to 3% by weight, preferably in quantities of 0.01 to 1% by weight and more particularly in quantities of 0.01 to 0.2% by weight, based on the total weight of the preparation.

According to the invention, the antiscale component (B) is selected from compounds corresponding to formula (I): 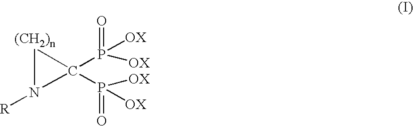
in which n is an integer of 3 to 5, R is hydrogen or a C₁₋₄ alkyl group and X is hydrogen, ammonium, substituted ammonium salts, such as mono-, di- or triethanolammonium salts, or alkali metal ions, such as sodium or potassium ions, at most three groups X simultaneously representing hydrogen.

2,2-Diphosphono-1-azacycloalkanes are excellent sequestering agents for polyvalent metal ions, more particularly for di- and trivalent metal ions. They are particularly suitable as complexing agents for alkaline earth metal ions.

In a most particularly preferred embodiment of the invention, azacycloheptane-2,2-diphosphonic acid or a physiologically compatible salt, for example, the sodium salt of azacycloheptane-2,2-diphosphonic acid, is used as the antiscale component in the oral and dental care preparations according to the invention.

The antiscale component (B) is used in the oral and dental care preparations according to the invention in quantities of 0.001 to 5% by weight, preferably in quantities of 0.01 to 3% by weight and more particularly in quantities of 0.05 to 2% by weight, based on the total weight of the preparation.

The third compulsory component (C) of the active-component combination according to the invention is the binder.

Binders which may be used for the purposes of the invention are xanthan gum and carboxymethyl cellulose. They are normally used in quantities of at least 0.01% by weight and more particularly in quantities of 0.05 to 2% by weight, based on the total weight of the preparation.

In a preferred embodiment of the invention, other binders or thickeners which have a consistency-adjusting effect and, in addition, prevent separation of the liquid and solid constituents may be added to the oral and dental care preparations according to the invention.

They are used in the preparations according to the invention in quantities of 0.1 to 5% by weight, preferably in quantities of 0.1 to 3% by weight and more particularly in quantities of 0.5 to 2% by weight.

Natural and/or synthetic water-soluble polymers, such as alginates, carrageenans, agar agar, guar gum, gum arabic, succinoglycan gum, guar flour, locust bean gum, tragacanth, karaya gum, pectins, hydroxyethyl cellulose or methyl hydroxypropyl cellulose, hydrophobicized celluloses, starch and starch ethers are used in accordance with the invention.

Water-soluble carboxyvinyl polymers (for example, Carbopol® types), polyvinyl alcohol, polyvinyl pyrrolidone and relatively high molecular weight polyethylene glycols (more particularly those with molecular weights of 10² to 10⁶ D) are also suitable binders of thickeners. Layer silicates and fine-particle silicas (Aerogel silicas and pyrogenic silicas) can also perform this function.

Preferred other binders or thickeners are the water-insoluble, non-derivatized celluloses marketed, for example, by J. Rettenmaier & Söhne under the names of Arbocel® and Vitacel®. In the context of the invention, water-insoluble means a solubility of less than 1% by weight in water at 20° C., i.e. less than 1% by weight of the cellulose is dissolved in 100 g of a saturated solution at 20° C.

Arbocel® CGP 5000, a highly viscous paste of cellulose powder with thixotropic properties, is a particularly effective thickener which, even when used in low concentrations, has strong consistency-imparting properties, is inert to ionic constituents and may readily be combined with other thickeners.

In a preferred embodiment of the invention, the oral and dental care preparations additionally contain surfactants or surfactant mixtures to improve their cleaning performance and foaming behavior. The surfactants or surfactant mixtures promote the rapid and complete dissolution and distribution of oral and dental care preparations in the mouth and simultaneously support the mechanical removal of plaque, particularly in places which are difficult to reach with a toothbrush. They also promote the incorporation of water-insoluble components, for example, flavoring oils, stabilize the dispersion of polishing agents and support the anticaries effect of fluorides.

In principle, anionic surfactants, zwitterionic and ampholytic surfactants, nonionic surfactants, cationic surfactants or mixtures of these compounds may be used as surfactants in toothpaste formulations. According to the invention, toothpastes preferably contain at least one surfactant from the group of anionic surfactants while mouthwashes preferably contain at least one surfactant from the group of nonionic surfactants.

The surfactant or surfactant mixture is normally used in the preparations according to the invention in a quantity of 0.1 to 10% by weight, preferably in a quantity of 0.3 to 7% by weight and more particularly in a quantity of 1 to 5% by weight, based on the total weight of the composition.

Anionic Surfactants

Suitable surfactants with a good foaming effect are anionic surfactants which also have a certain enzyme-inhibiting effect on the bacterial metabolism of plaque. Such anionic surfactants include, for example, alkali metal or ammonium salts, more particularly sodium salts, of C₈₋₁₈ alkanecarboxylic acids, of alkyl polyglycol ether sulfates containing 12 to 16 carbon atoms in the linear alkyl group and 2 to 6 glycol ether groups in the molecule, of linear alkane-(C₁₂₋₁₈)-sulfonates, sulfosuccinic acid monoalkyl-(C₁₂₋₁₈)-esters, sulfated fatty acid monoglycerides, sulfated fatty acid alkanolamides, sulfoacetic acid alkyl-(C₁₂₋₁₆)-esters, acyl sarcosines, acyl taurides and acyl isethionates containing 8 to 18 carbon atoms in the acyl group.

It is preferred to use at least one anionic surfactant, more particularly a sodium lauryl alkyl sulfate containing 12 to 18 carbon atoms in the alkyl group. One such surfactant is sodium lauryl sulfate which is commercially obtainable, for example, under the name of Texapon® K 1296.

Zwitterionic and Ampholytic Surfactants

In a preferred embodiment of the invention, zwitterionic and/or ampholytic surfactants may be used, preferably in combination with anionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethylammonium glycinates, for example, trimethyl ammonium glycinate, cocoalkyl dimethyl ammonium glycinate, N-acylaminopropyl-N,N-dimethyl ammonium glycinates, for example, cocoacyl aminopropyl dimethyl ammonium glycinate, and 2-alkyl-3-carboxymethyl-3-hydroxyethyl imidazolines containing 8 to 18 carbon atoms in the alkyl or acyl group and cocoacyl aminoethyl hydroxyethyl carboxymethyl glycinate. The fatty acid amide derivative known by the CTFA name of Cocamidopropyl Betaine is particularly preferred. Such products are commercially obtainable, for example, under the name of Tego-Betain® BL 215 and ZF 50 and Genagen® CAB.

Ampholytic surfactants are surface-active compounds which, in addition to a C₈₋₁₈ alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Besides ampholytic surfactants, quaternary emulsifiers are also suitable, those of the esterquat type, preferably methyl-quaternized difatty acid triethanolamine ester salts, being particularly preferred.

Nonionic Surfactants

According to the invention, nonionic surfactants are particularly suitable for supporting the cleaning effect. Particularly preferred nonionic surfactants are those selected from at least one of the following groups:
products of the addition of 2 to 30 mol ethylene oxide and/or 0 to 5 mol propylene oxide onto linear fatty alcohols containing 8 to 22 carbon atoms, onto fatty acids containing 12 to 22 carbon atoms and onto alkylphenols containing 8 to 15 carbon atoms in the alkyl group;
C_{12/18} fatty acid monoesters and diesters of products of the addition of 1 to 30 mol ethylene oxide onto glycerol;
glycerol monoesters and diesters and sorbitan mono- and diesters of saturated and unsaturated fatty acids containing 6 to 22 carbon atoms and ethylene oxide adducts thereof;
alkyl mono- and oligoglycosides containing 8 to 22 carbon atoms in the alkyl chain and ethoxylated analogs thereof
products of the addition of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate.
   Mixtures of compounds from several of these classes are also suitable.
products of the addition of 2 to 15 mol ethylene oxide onto castor oil and/or hydrogenated castor oil;
partial esters based on linear, branched, unsaturated or saturated C₆₋₂₂ fatty acids, ricinoleic acid and 12-hydroxystearic acid and glycerol, polyglycerol, pentaerythritol, dipentaerythritol, sugar alcohols (for example, sorbitol), sucrose, alkyl glucosides (for example, methyl glucoside, butyl glucoside, lauryl glucoside) and polyglucosides (for example, cellulose);
mono-, di- and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol according to DE 1165574 PS and/or mixed esters of C₆₋₂₂ fatty acids, methyl glucose and polyols, preferably glycerol and polyglycerol and
polyalkylene glycols.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products and are preferred for the purposes of the invention. They are homolog mixtures of which the average degree of alkoxylation corresponds to the ratio between the quantities of ethylene oxide and/or propylene oxide and substrate with which the addition reaction is carried out. C₁₂₋₁₈ fatty acid monoesters and diesters of adducts of ethylene oxide with glycerol are known as lipid layer enhancers for cosmetic preparations from DE 20 24 051 PS.

C₈₋₁₈ alkyl mono- and oligoglycosides, their production and their use are known from the prior art, for example, from U.S. Pat. No. 3,839,318, DE-A-20 36 472, EP-A-77 167 or WO-A-93/10132. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based. A particularly suitable alkyl (oligo)glycoside is one which corresponds to the formula RO(C₆H₁₀O)ₓ—H, where R is a C₁₂₋₁₄ alkyl group and x has a mean value of 1 to 4.

A particularly preferred surfactant used in the oral and dental care preparations according to the invention is the alkyl glycoside marketed by Cognis under the name of Plantacare 1200 UP (C₁₂₋₁₆ fatty alcohol-1,4-glucoside).

The oral and dental care preparations according to the invention additionally contain humectants to protect them against drying out and for adjusting their consistency and low-temperature stability. However, the humectants may also be used for suspension purposes and for influencing taste and luster.

The humectants typically used are toxicologically safe polyols such as, for example, sorbitol, xylitol, glycerol, mannitol, 1,2-propylene glycol or mixtures thereof. However, polyethylene glycols with molecular weights of 200 to 2,000 and more particularly in the range from 200 to 1,500 may also be used as humectant components in toothpastes.

A combination of several humectant components is preferably used. The combination of glycerol and/or sorbitol with 1,2-propylene glycol or polyethylene glycol may be regarded as particularly preferred.

The combination of xylitol with one or both of the humectants glycerol and sorbitol is another preferred combination which, besides its function as a humectant, also benefits from the anticariogenic properties of xylitol.

In cases where the oral and dental care preparations according to the invention are formulated as mouthwashes, they may additionally contain ethanol in quantities of 0 to 10% by weight and preferably in quantities of 1 to 5% by weight, based on the total weight of the preparation.

Depending on the type of product, the humectant or the mixture of humectants is present in the preparation as a whole in a quantity of 10 to 85% by weight, preferably in a quantity of 20 to 70% by weight and more particularly in a quantity of 30 to 50% by weight.

In another preferred embodiment, the preparation according to the invention additionally contains oil/fat and/or wax components. These components provide the preparations according to the invention with luster and flexibility, particularly when they are used in combination with surfactants. Natural, chemically modified and synthetic waxes, fats and oils may be used either individually or in combination for the purposes of the invention.

The oil/fat and wax components are used in a quantity of 0.1 to 10% by weight, preferably in a quantity of 0.5 to 5% by weight and more particularly in a quantity of 0.8 to 3% by weight, based on the preparation as a whole.

Oils/Fats

In the context of the invention, oils/fats are understood to be liquid or solid mono-, di- and triglycerides and also hydrocarbons and silicone oils which may be of natural or synthetic origin. According to the invention, the oil components are preferably selected from the group of triglycerides and/or paraffin oils which may be of vegetable, animal or synthetic origin.

Mono-, di- and triglycerides are the mono-, di- and triesters of fatty acids with glycerol, i.e. acyl glycerols. The glycerol may be esterified with the same or different fatty acids or fatty acid derivatives (for example, lecithin). The fatty acids are preferably C₆₋₃₀ fatty acids which may be saturated or unsaturated and branched or unbranched. Such fatty acids include inter alia products of the esterification of glycerol with naturally occurring fatty acids such as, for example, caproic acid, oenanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachic acid, behenic acid, lignoceric acid, cerotic acid, melissic acid, 2-ethylhexanoic acid, isotridecanoic acid, isostearic acid, palmitoleic acid, oleic acid, elaidic acid, petroselic acid, elaeostearic acid, erucic acid, linoleic acid, linolenic acid, arachidonic acid, clupanodonic acid, docosahexanoic acid and gadoleic acid and the technical mixtures thereof obtained, for example, in the pressure hydrolysis of natural fats and oils or in the dimerization of unsaturated fatty acids. The use of natural fats and oils such as, for example, bovine tallow, peanut oil, rapeseed oil, cottonseed oil, soybean oil, sunflower oil, palm oil, palm kernel oil, linseed oil, almond oil, castor oil, corn oil, olive oil, colza oil, sesame oil, cocoa butter and coconut fat and the like can be particularly suitable. Hydrogenated orhardened oils, for example, hydrogenated soybean oil, castor oil and peanut oil, may also be used.

Thinly and thickly liquid silicone oils or natural and synthetic hydrocarbons such as, for example, thinly or thickly liquid paraffin oils, isohexadecane, isoeicosane or polydecenes obtainable, for example, under the names of Emery® 3004, 3006, 3010, Ethylflo® or Nexbase® 2004G may also be used in accordance with the invention.

So-called inverse fats (for example, esters of vicinal tricarboxylic acids with C₆₋₃₀ fatty alcohols) or of glycerol trialkyl ethers may also be used.

Waxes

Waxes are understood to be natural or synthetic materials with the following properties: they are solid or fragile and hard in consistency, coarsely to finely crystalline, transparent or opaque and melt above 35° C. without decomposing. They are low in viscosity and non-stringing only slightly above their melting point and show highly temperature-dependent consistency and solubility.

Chemically, the most common representatives from the group of waxes are esters of fatty acids and higher fatty alcohols of animal and vegetable origin. Waxes suitable for use in accordance with the present invention are natural vegetable waxes such as, for example, jojoba oil, candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. It can also be of advantage to use hydrogenated waxes.

Natural waxes usable in accordance with the invention also include the mineral waxes, such as ceresine and ozocerite for example, or the petrochemical waxes, such as paraffin waxes (for example, Vaseline) and microwaxes. Other suitable wax components are chemically modified waxes such as, for example, montan ester waxes, sasol waxes and hydrogenated jojoba waxes. Synthetic waxes usable in accordance with the invention include, for example, wax-like polyalkylene waxes and silicone waxes.

Synthetic waxes, more particularly the esters of C₆₋₃₀ fatty acids with C₆₋₃₀ fatty alcohols which may be of natural or synthetic origin, may also be used in accordance with the invention. Both the fatty acid component and the fatty alcohol component may be linear or branched and saturated or mono- or poly-unsaturated.

The wax component is preferably selected from the group of vegetable or animal waxes and/or paraffin waxes or mixtures of these waxes.

Beeswax and jojoba oil are particularly preferred wax components.

In another preferred embodiment, the oral and dental care preparations according to the invention may also contain additional active components, such as anticaries components, remineralizing components, flavoring components and other antimicrobial agents and/or scale inhibitors or combinations of these active components.

Anticaries Components

Components suitable for controlling and preventing caries are, above all, fluorine compounds, preferably from the group of fluorides or monofluorophosphates in a quantity of 0.1 to 0.5% by weight fluorine. Suitable fluorine compounds are, for example, sodium fluoride, potassium fluoride, tin fluoride, disodium monofluorophosphate (Na₂PO₃F), dipotassium monofluorophosphate or the fluoride of an organic amino compound. Organic amine fluorides in the context of the invention are, for example, ammonium fluoride, cetylamine hydrofluoride and bis-(hydroxyethyl)-aminopropyl-N-hydroxyethyl octadecyl amine dihydrofluoride.

In one particularly preferred embodiment of the invention, sodium fluoride, ammonium fluoride or an organic amine fluoride is added to the oral and dental care preparation.

Antimicrobial Components

Suitable antimicrobial components are, for example, phenols, resorcinols, bisphenols, salicyl anilides and amides and halogenated derivatives thereof, halogenated carbanilides and p-hydroxybenzoic acid esters.

Among the antimicrobial components, those which inhibit the growth of plaque bacteria are particularly suitable. For example, halogenated diphenylethers, such as 2,4-dichloro-2′-hydroxydiphenylether, 4,4′-dichloro-2′-hydroxydiphenylether, 2,4,4′-tribromo-2′-hydroxydiphenylether, 2,4,4′-trichloro-2′-hydroxydiphenylether (triclosan), are suitable antimicrobial agents. Besides bromochlorophene, phenyl salicylic acid esters and 5-amino-1,3-bis-(2-ethylhexyl)-hexahydro-5-methylpyrimidine (hexetidine), zinc and copper ions also have antimicrobial activity, synergistic effects occurring in particular in combination with hexetidine and triclosan. Quaternary ammonium compounds such as, for example, cetyl pyridinium chloride, benzalkonium chloride, domiphenbromide and dequalinium chloride, may also be used. Octapinol, octenidines and sanguinarin have also been found to have antimicrobial activity. The antimicrobial components are preferably used in the preparations according to the invention in quantities of 0.01 to 1% by weight. In a particularly preferred embodiment, Irgacare® MP is used in a quantity of 0.01 to 0.3% by weight.

Scale Inhibitors

Scale is formed by mineral deposits which are very similar to natural dental enamel. In order to inhibit the formation of scale, substances which selectively engage in the formation of crystal nuclei and prevent nuclei already present from further growth are added to the oral and dental care preparations according to the invention. The substances in question are, for example, condensed phosphates which are preferably selected from the group of tripolyphosphates, pyrophosphates, trimetaphosphates or mixtures thereof. They are used in the form of their alkali metal or ammonium salts, preferably in the form of their sodium or potassium salts. Aqueous solutions of these phosphates typically show an alkaline reaction, so that the pH of the oral and dental care preparations according to the invention is optionally adjusted to a value of 7.5 to 9 by addition of acid. The acid may be selected, for example, from citric acid, phosphoric acid or acidic salts, for example, NaH₂PO₄. However, the required pH of the oral and dental care preparation may also be adjusted by addition of acidic salts of the condensed phosphates, i.e., for example, K₂H₂P₂O₇.

Mixtures of various condensed phosphates and/or hydrated salts of the condensed phosphates may also be used in accordance with the invention. Scale inhibitors are normally used in the preparations according to the invention in quantities of 0.1 to 5% by weight, preferably in quantities of 0.1 to 3% by weight and more particularly in quantities of 0.1 to 2% by weight.

Other suitable scale inhibitors are organophosphonates, such as 1-hydroxyethane-1,1-diphosphonate (Na salt) and zinc citrate.

Remineralizing Components

The preparations according to the invention preferably also contain components which promote remineralization of the dental enamel and are capable of closing dental lesions. These components are normally present in a total quantity of 0.1 to 10% by weight, preferably 0.1 to 5% by weight and more particularly 0.1 to 3% by weight. The remineralizing components include, for example, fluorides, phosphate salts of calcium such as, for example, calcium glycerophosphates, calcium hydrogen phosphate, hydroxylapatite, fluoroapatite, F-doped hydroxylapatite, dicalcium phosphate dihydrate and calcium fluoride. However, magnesium salts such as, for example, magnesium sulfate, magnesium fluoride or magnesium monofluorophosphate also have a remineralizing effect.

Flavoring Components

The preparations according to the invention preferably contain flavoring components including, for example, sweeteners and/or flavoring oils. Suitable sweeteners are, for example, saccharinates (more particularly sodium saccharinate), cyclamates (more particularly sodium cyclamate) and sucrose, lactose, maltose or fructose. Suitable flavoring oils are any of the natural and synthetic flavors typically used in oral and dental care preparations. Natural flavors may be used both in the form of the essential oils isolated from the drugs and in the form of the individual components isolated therefrom. The dentifrice should preferably contain at least one flavoring oil from the group consisting of peppermint oil, spearmint oil, anise oil, Japanese anise oil, caraway oil, eucalyptus oil, fennel oil, cinnamon oil, clove oil, geranium oil, sage oil, pimento oil, thyme oil, marjoram oil, basil oil, citrus oil, gaultheria oil or one or more components of these oils isolated from them or synthetically produced. The most important components of the oils mentioned are, for example, menthol, carvone, anethol, cineol, eugenol, cinnamaldehyde, caryophyllene, geraniol, citronellol, linalool, salvia, thymol, terpinene, terpineol, methyl chavicol and methyl salicylate. Other suitable flavors are, for example, menthyl acetate, vanillin, ionone, linalyl acetate, rhodinol and piperitone.

Where the oral and dental care preparations according to the invention are formulated as toothpastes, tooth gels or liquid transparent tooth cleaning preparations, they contain at least one abrasive as another constituent.

The abrasives are used for mechanical removal of the uncalcified plaque and, ideally, should lead to polishing of the tooth surface (polishing effect) with minimal abrasion (abrasive effect) and damage to the enamel and the dentine. The abrasion behavior of the abrasives is largely determined by their hardness, particle size distribution and surface structure. Accordingly, when choosing suitable abrasives, particular preference will be given to those which combine high cleaning performance with minimal abrasion. The abrasives mainly used nowadays are substances with small particle sizes which are largely free from sharp corners and edges.

Water-soluble inorganic materials are normally used as abrasives or polishes. It is of particular advantage to use very fine-particle polishes with a mean particle size of 1 to 200 μm, preferably 1 to 50 μm and more particularly 1 to 10 μm.

In principle, the polishes according to the invention may be selected from silicas, aluminium hydroxide, aluminium oxide, silicates, organic polymers or mixtures thereof. However, so-called metaphosphates and calcium-containing polishing components may also be present in the preparations according to the invention in quantities of up to 5% by weight.

In a preferred embodiment of the invention, silicas are used as polishes in toothpastes or liquid tooth cleaning preparations. Basically, silica polishes are divided into silica gels, silica hydrogels and precipitated silicas. Precipitated silicas and silica gels are particularly preferred for the purposes of the invention because they can be widely varied in their production and are particularly compatible with fluoride components. In addition, they are particularly suitable for the production of gel or liquid tooth creams.

Silica gels are obtained by reacting sodium silicate solutionswith strong aqueous mineral acids to form a hydrosol, ageing to form the hydrogel, washing and drying. If drying is carried out under moderate conditions to a water content of 15 to 35% by weight, the so-called silica hydrogels described, for example, in U.S. Pat. No. 4,153,680 are obtained. Drying of these silica hydrogels to water contents below 15% by weight results in irreversible shrinkage of the previously loose structure of the hydrogel to the dense structure of the so-called xerogel. Silica xerogels are described, for example, in U.S. Pat. No. 3,538,230.

A second particularly suitable group of silica polishing agents are the precipitated silicas. Precipitated silicas are obtained by precipitation of silica from dilute alkali metal silicate solutions by addition of strong acids under conditions which preclude aggregation to the sol and gel. Suitable processes for the production of precipitated silicas are described, for example, in DE-OS 25 22 586 and in DE-OS 31 14 493. A particularly suitable precipitated silica for the purposes of the present invention is that produced in accordance with DE-OS 31 14 493 which has a BET surface of 15 to 110 m²/g, a particle size of 0.5 to 20 μm (at least 80% by weight of the primary particles should be below 5 μm in size) and a viscosity in the form of a 30% glycerol/water (1:1) dispersion of 30 to 60 Pa·s (20° C.) and which is used in a quantity of 10 to 20% by weight, based on the toothpaste. In addition, particularly suitable precipitated silicas of this type have rounded corners and edges and are commercially obtainable under the name of Sident® 12 DS (Degussa).

Other precipitated silicas of this type are Sident 8 (Degussa) and Sorbosil AC 39 (Crosfield Chemicals). These silicas are distinguished by a weaker thickening effect and a slightly larger mean particle size of 8 to 14 μm for a specific BET surface of 40 to 75 m²/g and are particularly suitable for liquid tooth creams. These should have a viscosity (25° C., shear rate D=10 s⁻¹) of 10 to 100 Pa·s.

In addition, silicas of theZeodent®type (HuberCorp.), theTixosil®type (Rhodia) and other Sorbosil types may be used in the preparations according to the invention. Zeodent® 113, Tixosil® 123 and 73 and Sorbosil® AC 33 are particularly preferred.

By contrast, toothpastes which have a distinctly higherviscosityof more than 100 Pa s (25° C., D=10 s⁻¹) require a sufficiently high percentage of silicas with a particle size below 5 μm, preferably at least 3% by weight silica with a particle size of 1 to 3 μm. Accordingly, besides the precipitated silicas mentioned, even finer so-called thickening silicas with a BET surface of 150 to 250 m²/g are added to such toothpastes. Examples of commercial products which meet the stated requirements are, in particular, Sipernat® 22Ls or Sipernat® 320 DS (products of Degussa).

A preferred aluminium oxide polish is a weakly calcined alumina with a content of at least 10% by weight of α-aluminium oxide of various, so-called γ-aluminium oxide modifications.

Suitable weakly calcined aluminas are produced by calcination from aluminium hydroxide. Aluminium hydroxide is converted by calcination into α-Al₂O₃ which is thermodynamically stable at temperatures above 1200° C. The thermodynamically unstable Al₂O₃ modifications occurring at temperatures between 400 and 1,000° C. are known as gamma forms (cf. Ullmann, Enzyclopädie der technischen Chemie, 4th Edition (1974), Vol. 7, page 298). By selecting the appropriate calcination temperature and calcination time, the degree of calcination, i.e. the conversion into the thermodynamically stable α-Al₂O₃, can be adjusted as required. Weak calcination gives an alumina with a γ-Al₂O₃ content which is lower, the higher the calcination temperature and the longer the calcination time. Weakly calcined aluminas differ from pure α-Al₂O₃ in a lower hardness of the agglomerates, a higher specific surface and larger pore volumes.

The dentine abrasion (RDA) of the relatively weakly calcined aluminas to be used in accordance with the invention with a γ-Al₂O₃ content of 10 to 50% by weight is only 30 to 60% of the dentine abrasion of a highly calcined pure α-Al₂O₃ (as measured in a standard toothpaste containing 20% by weight alumina as sole polishing component).

In contrast to α-Al₂O₃, γ-Al₂O₃ can be colored red with an aqueous/ammoniacal solution of Alizarin S (1,2-dihydroxy-9,10-anthraquinone-4-sulfonic acid). The degree of “dyeability” may be selected as a measure of the degree of calcination or the percentage content of δ-Al₂O₃ in a calcined alumina: ca, 1 g Al₂O₃, 10 ml of a solution of 2 g/l Alizarin S in water and 3 drops of an aqueous 10% by weight solution of NH₃ are introduced into a test tube and briefly boiled. The Al₂O₃ is then filtered off, washed, dried and examined under a microscope or calorimetrically evaluated.

Suitable weakly calcined aluminas with a γ-Al₂O₃ content of 10 to 50% by weight can be colored pale to deep pink by this process.

Aluminium oxide particles with various degrees of calcination, particle finenesses and bulk densities are commercially available, for example, the “Poliertonerden” of Giulini-Chemie or ALCOA.

A particularly suitable quality “Poliertonerde P10 feinst” has an agglomerate size below 20 μm, a mean primary crystal size of 0.5 to 1.5 μm and a bulk density of 500 to 600 g/l.

According to the invention, silicates may also preferably be used as polishing components. In modern practice in particular, silicates are used as abrasives. Examples of silicates suitable for use in accordance with the invention are aluminium silicates and zirconium silicates. In one particular embodiment, sodium aluminium silicate with the empirical formula Na₁₂(AlO₂)₁₂(SiO₂)₁₂×7H₂O, for example, synthetic zeolite A, may be suitable as a polish.

Examples of water-insoluble metaphosphates according to the invention are, above all, sodium metaphosphate, calcium phosphate such as, for example, tricalcium phosphate, calcium hydrogen phosphate, calcium hydrogen phosphate dihydrate and calcium pyrophosphate.

In addition, magnesium carbonate, magnesium hydrogen phosphate, trimagnesium phosphate or sodium hydrogen carbonate may also be used as a polishing component in accordance with the invention, particularly in the form of a mixture with other polishes.

The oral and dental care preparations according to the invention may preferably contain a number of other components, including inter alia:
vitamins, for example, retinol, biotin, tocopherol, ascorbic acid and derivatives thereof (for example, esters, salts);
pigments, for example, titanium dioxide or zinc oxide;
colored pigment particles, for example, the colored silica particles commercially obtainable, for example, underthe names of Sorbosil® BFG 51, BFG 52 and BFG 53 or Sorbosil® 2352. Mixtures of differently colored pigment particles may also be used. Silica gel particles such as these, for example, colored bright orange, red or blue, may be present in the preparations according to the invention in quantities of 0.1 to 1.0% by weight;
bleaching agents;
dyes;
pH adjusters and buffers, for example, sodium citrate, sodium bicarbonate or potassium and sodium phosphates,
sodium benzoate;
wound-healing and inflammation-inhibiting components such as, for example, allantoin, urea, panthenol, azulene or camomile extract, acetyl salicylic derivatives, thiocyanate; hydrogen peroxide;
zinc and manganese sulfate.

Particulars of other optional components and the quantities in which they are used can be found in the relevant handbooks known to the expert, for example, K. Schrader Grundlagen und Rezepturen der Kosmetika, 2nd Edition, 1989, Dr. Alfred Hüthig Verlag, Heidelberg, and W. Umbach Kosmetik: Entwicklung, Herstellung und Anwendung kosmetischer Mittel, 2nd Edition, Thieme Verlag, Stuttgart 1995.

The oral and dental care preparations according to the invention are meant to be regarded as a daily cosmetic, nontherapeutic treatment. The teeth are cleaned and freed from discoloration and food remains which makes their cosmetic appearance more attractive and pleasing.

Accordingly, in a second embodiment, the present invention relates to a nontherapeutic process for reducing and preventing discoloration on human teeth, in which an oral and dental care preparation containing an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components
   is used for daily oral and dental care.

Accordingly, in a third embodiment, the present invention relates to a nontherapeutic process for inhibiting scale and plaque, in which an oral and dental care preparation containing an active-component combination of at least one cationic antibacterial component,
(A) at least one antiscale component selected from the group of azacycloalkane diphosphonic acids or physiologically compatible salts thereof and
(B) at least one binder selected from xanthan gum, carboxymethyl cellulose or a mixture of these two components
   is used for daily oral and dental care.

In a fourth embodiment, the present invention relates to the nontherapeutic use of an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components
   for reducing and preventing discoloration on human teeth.

In a fifth embodiment, the present invention relates to the nontherapeutic use of an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components,
   for inhibiting scale and plaque.

The following EXAMPLES are intended to illustrate the invention.

### EXAMPLES

1) Whitening Effect or Reduction of Discoloration on Teeth

The discoloration-reducing or whitening effect was demonstrated in vitro on bovine teeth using a special staining and measuring process.

Reagents:

Bag tea/East Frisian mixture

Materials and apparatus

Grey wax (Kerr)

Sample holders

Plastic coloring wheel (diameter 22 cm)

Plastic tray for coloring wheel (18×5×3.5)

Apparatus with motor for moving at most 5 wheels

Dr. Lange “Microcolor” colorimetric measuring instrument

Test description

The bovine incisors are cut to form flat slabs of the labial sides with an area of 7×7 mm. The slabs are affixed with the wax to the middle of the sample holders so that only the enamel surface is exposed. The enamel surface is uniformly rubbed with abrasive paper (grade 600) to eliminate unevenness and impurities.

The Dr. Lange Microcolor calorimetric measuring instrument is used for all investigations.

The sample was illuminated with light of Standard Light Type D65 (=daylight). The entire slab surface was measured. Stray light was prevented from entering during the measurement by screening.

The lightness value L* according to DIN 6174 was used as the test barometer for evaluating the discoloration of the sample or the discoloration-inhibiting effect.

4 Slabs per group were placed in the plastic tray with the enamel side up and covered either with 40 g mouthwash or with the centrifugate of a suspension of 10 g tooth cream in 40 g distilled water. The treatment with active-component solution was carried out twice daily, morning and evening.

After a contact time of 5 mins. at room temperature, the slabs were thoroughly rinsed in running distilled water, then placed on the coloring wheel and moved at 2 r.p.m. through a coloring bath which was freshly prepared twice daily by brewing 3 g bag tea in 300 ml water. The tea was then allowed to draw for 10 minutes. Before each bag change, the slabs were rinsed with distilled water and, thereafter, were additionally rubbed dry with cellulose to remove loosely adhering coatings. This treatment scheme was carried out for 10 days.

After the last coloring with tea on day 10, the lightness value L* of the samples was measured and the difference in relation to the starting values was determined and averages were calculated.

2) Scale- and Plaque-Inhibiting Effect

The scale- and plaque-inhibiting effect was demonstrated by determining the crystal growth of hydroxylapatite.

By adding hydroxylapatite to an oversaturated Ca phosphate solution, crystallization was initiated in accordance with the following equation:
5Ca²⁺+3HPO₄²⁻+H₂O→Ca²⁺₅(PO₄³⁻)₃OH+4H⁺

The crystal growth rate, which can be influenced by active components, was determined by titration with an alkali.

Apparatus:

thermostat
magnetic stirrer and rod
autoburette 25 ml (ABU 80 Radiometer)
pH meter (82 Radiometer)
pH electrode (N61 Schott)
titrator (TTT80 Radiometer)
servograph (Rec 80 Radiometer)
centrifuge, centrifuge glasses
glass beakers: 800 ml double-walled 200 ml
pipette: 10 ml
measuring cylinder: 500 ml
burette: 50 ml
   General Description:

400 ml of 0.0008 molar KH₂PO₄ solution and 45 ml of 0.012 molar CaCl₂ solution were poured into a reaction vessel thermostatted to 37° C. The combined solutions were then titrated to pH 7.4 with 0.05 molar KOH. When the pH had remained stable for 15 mins., 100 mg apatite were added and the whole was titrated with stirring for 2 hours at pH 7.4. The quantity of KOH consumed was recorded by servograph as a function of time.

Preparation of the Preparations:

Toothpaste slurry consisting of 20 g toothpaste and 80 g deionzed water was thoroughly stirred for 15 mins and then centrifuged. 10 ml of this clear solution were added to the Ca phosphate solution and a pH of 7.4 was adjusted before the addition of 100 mg apatite.

In the testing of mouthwash, 10 g mouthwash were added to the Ca phosphate solution.

Test Evaluation:

The curve without toothpaste was evaluated as the zero curve with 0% inhibition. The KOH consumption of the zero curve was read off after 2 hours and compared with the consumption of toothpaste solution.

$$\% I = \frac{AN - AZ}{AN} \times 100$$

% I=% inhibition
AN=consumption of the zero curve
AZ=consumption of the toothpaste solution.

The following test mixtures were prepared (all quantities=% by weight, unless otherwise indicated):

TABLE 1

| RAW MATERIAL | A | B | C | D |
|---|---|---|---|---|
| Chlorhexidine digluconatet¹ (AS) | 0.03 | 3 | 0.03 | 0.03 |
| Alkyl polyglucoside² | 0.1 | 0.1 | 0.1 | 0.1 |
| Keltrol F³ | — | 2 | — | 0.2 |
| AHP 100⁴ | — | — | 0.2 | 0.2 |
| Tagat CH 60⁵ | 0.2 | 0.2 | 0.2 | 0.2 |
| Ethanol 99.8% | 5.0 | 5 | 5.0 | 5 |
| Flavor | 0.15 | 0.15 | 0.15 | 0.15 |
| Sodium fluoride | 0.045 | 0.045 | 0.045 | 0.045 |
| Xylitol | 1.0 | 1.0 | 1.0 | 1.0 |
| Sodium saccharin | 0.03 | 0.03 | 0.03 | 0.03 |
| Polydiol 400⁶ | — | 1.0 | — | 1.0 |
| Sorbitol 70% | 0.9 | 0.9 | 0.9 | 0.9 |
| Sodium hydroxide | — | — | 0.063 | 0.063 |
| Discoloration | yes | yes | yes | no |
| Whitening effect | no | no | no | yes |
| Change in lightness (delta L*) | −23.0 | −28.3 | −24.6 | 5.1 |
| by comparison with zero | | | | |
| sample (10 days) | | | | |
| Antiscale effect | — | — | 72% | 75% |
| Antibacterial effect | good | good | good | good |

Result:

Only sample D, i.e. the formulation with the three-component combination of AHP, xanthan gum (Keltrol F) and chlorhexidine digluconate, has a whitening effect.

TABLE 2

| RAW MATERIAL | D1 | D2 | D3 | D4 | D5 | D6 |
|---|---|---|---|---|---|---|
| Chlorhexidine | 0.03 | 0.03 | 0.03 | 0.03 | 0.03 | 0.03 |
| digluconate (AS) | | | | | | |
| Alkyl | 0.1 | 0.1 | 0.1 | 0.1 | 0.1 | 0.1 |
| polyglucoside | | | | | | |
| Keltrol F | 0.1 | 0.05 | 0.02 | 0.1 | 0.05 | 0.02 |
| AHP 100 | 0.2 | 0.2 | 0.2 | 0.1 | 0.1 | 0.1 |
| Tagat CH60 | 0.2 | 0.2 | 0.2 | 0.2 | 0.2 | 0.2 |
| Ethanol 99.8% | 5.0 | 5 | 5 | 5.0 | 5.0 | 5 |
| Flavor | 0.15 | 0.15 | 0.15 | 0.15 | 0.15 | 0.15 |
| Sodium fluoride | 0.045 | 0.045 | 0.045 | 0.045 | 0.045 | 0.045 |
| Xylitol | 1.0 | 1 | 1 | 1.0 | 1.0 | 1 |
| Sodium saccharin | 0.03 | 0.03 | 0.03 | 0.03 | 0.03 | 0.03 |
| Polydiol 400 | 1.0 | 1 | 1 | 1.0 | 1.0 | 1 |
| Sorbitol 70% | 0.9 | 0.9 | 0.9 | 0.9 | 0.9 | 0.9 |
| Sodium hydroxide | 0.063 | 0.063 | 0.063 | 0.03 | 0.0315 | 0.0315 |
| Formulation | O.K. | precip- | precip- | O.K. | precip- | precip- |
| stability | | itation | itation | | itation | itation |
| Antibacterial | good | — | — | good | — | — |
| effect | | | | | | |
| Antiscale effect | 74% | — | — | 55% | — | — |
| Whitening effect | yes | — | — | yes | — | — |
| Change in | 5.9 | — | — | 0.7 | — | — |
| lightness (delta | | | | | | |
| L*) by comparison | | | | | | |
| with zero sample | | | | | | |
| (10 days) | | | | | | |

Result:

Formulation stability is only guaranteed above a concentration of 0.1% Keltrol (xanthan gum), precipitation occurring with lower Keltrol concentrations.

TABLE 3

| RAW MATERIAL | E1 | E2 |
|---|---|---|
| Chlorhexidine digluconate (AS) | 0.03 | 0.03 |
| Alkyl polyglucoside | 0.1 | 0.1 |
| Carboxymethyl cellulose | — | 0.2 |
| AHP 100 | 0.2 | 0.2 |
| Tagat CH60 | 0.2 | 0.2 |
| Ethanol 99.8% | 0 | 5 |
| Flavor | 0.15 | 0.15 |
| Sodium fluoride | 0.045 | 0.045 |
| Xylitol | 1 | 1 |
| Sodium saccharin | 0.03 | 0.03 |
| Sorbitol 70% | 0.9 | 0.9 |
| Sodium hydroxide | — | 0.063 |
| Discoloration | yes | no |
| Whitening effect | no | yes |
| Change in lightness (delta L*) | −8.2 | 5.6 |
| by comparison with zero sample (10 days) | | |
| Antiscale effect | — | 75% |
| Antibacterial effect | — | good |

Result:

With this composition, too, a discoloration-inhibiting effect is only obtained by addition of the polymer component carboxymethyl cellulose.

The following raw materials and commercial products were used in the EXAMPLES:
1 Marketed by Degussa as a 20% solution
2 INCI name: Lauryl Glucoside; AS: 50-53%, marketed by Cognis
3 INCI name: Xanthan Gum, marketed by CP Kelco
4 INCI name: Disodium Azacycloheptane Diphosphonate, marketed by Albright & Wilson
5 INCI name: PEG-60 Hydrogenated Castor Oil, marketed by Goldschmidt

1. An oral and dental care preparation containing an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components.

2. The oral and dental care preparation as claimed in claim 1, characterized in that component (B) is azacycloheptane-2,2-diphosphonic acid or a physiologically compatible salt of this compound.

3. The oral and dental care preparation as claimed in claim 1, characterized in that the antibacterial component (A) is selected from the cationically derivatized bisbiguanides.

4. The oral and dental care preparation as claimed in claim 1, characterized in that the antibacterial component (A) is a physiologically compatible salt of chlorhexidine or alexidine.

5. The oral and dental care preparation as claimed in claim 4, characterized in that the antibacterial component (A) is chlorhexidine digluconate.

6. The oral and dental care preparation as claimed in claim 1, characterized in that the antibacterial component (A) is present in a quantity of 0.001 to 3% by weight, based on the total weight of the preparation.

7. The oral and dental care preparation as claimed in claim 1, characterized in that the antiscale component (B) is used in a quantity of 0.001 to 5% by weight, based on the total weight of the preparation.

8. The oral and dental care preparation as claimed in claim 1, characterized in that the binder (C) or the binder mixture is present in a quantity of at least 0.01% by weight, based on the total weight of the preparation.

9. The oral and dental care preparations as claimed in claim 1, characterized in that at least one nonionic surfactant is also present in the preparation.

10. The oral and dental care preparation as claimed in claim 9, characterized in that the nonionic surfactant is selected from the group of alkyl polyglycosides.

11. The oral and dental care preparation as claimed in claim 1, characterized in that they additionally contain at least one fluoride source.

12. The oral and dental care preparation as claimed in claim 11, characterized in that the fluoride source is selected from the group consisting of sodium fluoride, ammonium fluoride or an organic amine fluoride.

13. The oral and dental care preparations as claimed in claim 1, further comprising at least one additional binderorthickener selected from the group consisting of alginates, carageen, agar agar, guar gum, gum arabic, succinoglycan gum, guar flavor, locust bean gum, tragacanth, karaya gum, pectins, hydroxyethyl cellulose, methyl hydroxypropyl cellulose, hydrophobicized celluloses, starch and starch ethers.

14. The oral and dental care preparation as claimed in claim 1, further comprising atleastone additional binderorthickenerselected from the group consisting of carboxyvinyl polymers, polyvinyl alcohol, polyvinyl pyrrolidine, polyethylene glycols having a molecular weight between 10² and 20⁶ D, layer silicates, fine particle silicas such as Aerogel silicals and pyrogenic silicas and water-insoluble, non-derivatized celluloses.

15. The oral and dental care preparation as claimed in claim 1, characterized in that at least one anionic surfactant is also present in the preparation.

16. The oral and dental care preparation as claimed in claim 1, characterized in that at least one zwitterionic surfactant and at least one ampholytic surfactant are also present in the preparation.

17. A nontherapeutic process for reducing and preventing discoloration on human teeth, characterized in that an oral and dental care preparation containing an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components is applied to the teeth.

18. A nontherapeutic process for inhibiting scale and plaque on human teeth, characterized in that an oral and dental care preparation comprising an active-component combination of
(A) at least one cationic antibacterial component,
(B) at least one antiscale component selected from the group consisting of azacycloalkane diphosphonic acids or physiologically compatible salts thereof and
(C) at least one binder selected from xanthan gum, carboxymethyl cellulose or a mixture of these two components is used for daily oral and dental care.

19. The nontherapeutic process of claim 17 for reducing and preventing discloloration on human teeth, characterized in that the binder (C) or the binder mixture is present in a quantity of at least 0.01% by weight, based on the total weight of the preparation.

20. The nontherapeutic process of claim 17, characterized in thatthe oral and dental care preparation comprising an active-component combination of
(A) at least one cationic antibacterial component,
(B) at least one antiscale component selected from the group of azacycloalkane diphosphonic acids or physiologically compatible salts thereof and
(C) at least one binder selected from xanthan gum, carboxymethyl cellulose or a mixture of these two components, is employed in oral and dental care preparations applied to the teeth.
